(19) United States
(12) Patent Application Publication
(10) Pub. No.: US 2016/0073934 A1
(43) Pub. Date: Mar. 17, 2016

(54) METHOD AND APPARATUS FOR MONITORING DYNAMIC STATUS OF A BODY

(71) Applicant: DORSAVI PTY LTD, Melbourne East, Victoria (AU)

(72) Inventors: Daniel Matthew Ronchi, Victoria (AU); Andrew James Ronchi, Victoria (AU); Edgar Charry, Victoria (AU); Wenzheng Hu, Victoria (AU); Aakanksha Chhikara, Victoria (AU)

(73) Assignee: dorseVi Pty Ltd., Victoria (AU)

(21) Appl. No.: 14/784,565

(22) PCT Filed: Apr. 14, 2014
(86) PCT No.: PCT/AU2014/000426

§ 371 Date: Oct. 14, 2015

(30) Foreign Application Priority Data
Apr. 15, 2013 (AU) 2013901306

(51) Int. Cl. A61B 5/11; A61B 5/00

(52) U.S. Cl. CPC A61B 5/112; A61B 5/7225; A61B 5/1121; A61B 5/7246; A61B 5/486; A61B 2562/0219; A61B 2562/0223

(57) ABSTRACT

Apparatus is disclosed for monitoring, measuring and/or estimating dynamic status of a body part of a vertebral mammal. The apparatus includes at least one kinematics sensor for measuring and for providing data for comparison to a first frame of reference data indicative of the dynamic status of the body part. The apparatus also includes a memory device adapted for storing the sensor data and the first frame of reference data and a processor adapted for processing the sensor data to evaluate a dynamic signature associated with the body part that correlates to the first frame of reference data. A method for monitoring, measuring and/or estimating dynamic status of a body part of a vertebral mammal is also disclosed.

### CROSS-REFERENCE TO RELATED APPLICATIONS

The present invention is related to the following patent applications assigned to the present applicant, the disclosures of which are incorporated herein by cross reference.

AU2012903399 filed on 7 Aug. 2012 and entitled Method and apparatus for measuring reaction forces.

AU2012904946 filed on 9 Nov. 2012 and entitled Method and apparatus for monitoring deviation of a limb.

### TECHNICAL FIELD

The present invention relates to a method and apparatus for monitoring, diagnosing, measuring and/or providing feedback on dynamic status of a body part of a vertebral mammal including musculoskeletal status. Musculoskeletal status may manifest while performing physical activities and/or movements including activities and/or movements such as walking, running, sprinting, hopping, landing, squatting and/or jumping. Some activities may include movements of limbs of interest including legs. Other activities such as playing a game of tennis may include movement of limbs of interest including arms.

The method and apparatus of the present invention may be useful for measuring and/or providing feedback on any dynamic or kinematic activity including any activity that includes vertical and/or horizontal movement, rotational and translational forces in 3 dimensions (3D), timing of forces and/or movements, accelerations, velocities, impact and/or vibration of a body or body part of the mammal. Data obtained from the dynamic or kinematic activity may be used to gauge dynamic status and/or musculoskeletal function of the mammal's body or body part. Moreover patterns of movement associated with a dynamic or kinematic activity may be defined and used as a reference to determine whether and when a mammal is moving normally or abnormally. This may help to evaluate whether or not a material change in dynamic status of the body or body part has taken place.

### BACKGROUND OF INVENTION

Injuries to the body including injuries to musculoskeletal parts of the body are not uncommon and may be painful events for recreational and elite sports-persons. Following an injury to the body it may be desirable to establish dynamic status of the body to determine rehabilitation status of the body and fitness of a subject to return to active duty including fitness to “return to play” (RTP).

The method and apparatus of the present invention may be used in elite sports applications such as change of direction (COD) running, acceleration and deceleration activities and hopping and/or landing, wherein relatively normal patterns of movement may be defined and used as a reference. That reference may be used to detect abnormal patterns which may indicate that the subject is not fit to return to play.

A number of mechanical, physiological and/or biomechanical changes may occur during the abovementioned activities and/or movements. Different patterns of movement such as gait patterns may be associated with forces experienced by various body parts or limbs. For example, each time that a body part or limb such as a foot collides with a surface such as the ground, a range of forces exerted during each collision may be measured to produce a cluster of data including magnitudes, directions and/or timings of accelerations. The data associated with a particular pattern of movement performed by a subject may reflect a pattern of movement or “dynamic signature” that may be unique to that subject.

By capturing a subject's pattern or movement or “dynamic signature” prior to an injury it may be possible to use the dynamic signature as a control reference to detect a change of status of the body following an injury, including status of rehabilitation of the body during healing to determine fitness of the subject to return to a physical activity such as sport.

Forces may also be measured on a whole body such as the body of a subject landing on a water or snow surface. This may have implications for assessing ski jumpers landing on a snow surface. In other examples forces may be measured on a worker's wrist/hand striking a surface in order to help align parts, such as a vehicle assembly worker striking a die component to push it into place with possible implications for assessing workplace injuries and fitness to return to work after an injury.

Ground Reaction Forces (GRF) have traditionally been measured by force plates fixed on the floor. However such measurements may constrain assessment and analysis to laboratory conditions. Use of force plates even when used outdoors creates an artificial environment as the subject will typically modify their natural gait pattern in order to land on the force plate. Applicant's AU2012903399 discloses use of sensors such as MEMS accelerometers on a tibia to measure tibial peak acceleration and determine peak vertical GRF in activities such as jogging and running in outdoor environments.

The present invention may alleviate the disadvantages of the prior art and/or may improve accuracy and/or validity and/or functionality and/or availability of kinematics data. The present invention may provide a facility to capture a mammal's unique pattern of movement pre and post injury. The present invention may also provide a facility to measure injury and rehabilitation status of a mammal in virtually any setting, out in the field.

The present invention may measure kinematics related data such as acceleration(s) and/or angular rate of change and/or magnetic field in one or more dimensions (eg. 3D), and may estimate corresponding GRFs and correlate these to amplitude, direction and/or timing of GRFs measured by force platforms. Other data may include measurements of run time, stride rate (cadence), speed, peak accelerations and load rate. The data is reported to assist with assessment of movement patterns in rehabilitation and Return to Play (RTP) protocols.

For example, the RTP protocols may include applications such as deceleration tests wherein a player runs and then comes to a forced stop, change of direction tests wherein a player runs and then changes direction and different types of hopping tests. The hopping tests may include Ground Hop (hop on the same spot on one leg), Hop and Stick (hop forwards over a cone and land on one leg), Hop Medial (hop laterally on the opposite leg of the direction of movement over a cone), Hop Lateral (hop laterally on the same leg of the direction of the movement over a cone), Hop cut (hop on one leg forwards and then hop sideways landing on the same leg). These tests may provoke or establish possible impairments in movement and functional activity suggesting an issue, injury or imbalance with musculoskeletal structure (refer FIG. 4). In some applications an accelerometer may be placed on a medial part of the tibia (refer FIG. 1) and may measure magnitude, direction and timing of a limb's contact with respect to a ground surface.

A reference herein to a patent document or other matter which is given as prior art is not to be taken as an admission that that document or matter was known or that the information it contains was part of the common general knowledge in Australia or elsewhere as at the priority date of any of the disclosure or claims herein. Such discussion of prior art in this specification is included to explain the context of the present invention in terms of the inventor's knowledge and experience.

Throughout the description and claims of this specification the words “comprise” or “include” and variations of those words, such as “comprises”, “includes” and “comprising” or “including, are not intended to exclude other additives, components, integers or steps.

### SUMMARY OF INVENTION

According to one aspect of the present invention there is provided apparatus for monitoring, measuring and/or estimating dynamic status of a body part of a vertebral mammal, said apparatus including:
at least one kinematics sensor for measuring relative to a first frame of reference data indicative of said dynamic status of said body part and for providing said data;
a memory device adapted for storing said data; and
a processor adapted for processing said data to evaluate a dynamic signature associated with said body part that correlates to said data.

The kinematics sensor may include an acceleration sensor for measuring acceleration of the body part relative to the first frame of reference and for providing data indicative of the acceleration. The acceleration sensor may include at least one inertial sensor. The acceleration sensor may be adapted for measuring acceleration along one or more orthogonal axes.

The kinematics sensor may include a rotation sensor for measuring rotation of the body part around one or more orthogonal axes relative to the first frame of reference and for providing data indicative of the rotation. The rotation sensor may include a gyroscope. The kinematics sensor may include a magnetic field sensor for measuring magnetic field around the body part and for providing data indicative of the magnetic field.

A dynamic signature may be measured prior to an injury to serve as a control reference. A dynamic signature may be measured following an injury to enable a material change in dynamic signature to be detected. The processor may be adapted to execute an algorithm for evaluating a change in dynamic signature of the body part relative to the control reference.

The algorithm may combine 3D inertial sensor data including accelerometer, gyroscope and/or magnetometer data. The algorithm may be adapted to transform the data from the first frame of reference to a second frame of reference in which the body part performs a movement. The algorithm may transform the acceleration data from a sensor to a global frame perspective or frame of reference. Data may be transformed from a sensor to the global frame of reference in applications such as running or walking in which the subject moves relative to a global frame.

The body part of the mammal may include legs and the apparatus may be adapted to monitor rotation components associated with the legs. Respective sensors may be applied to the legs of the mammal.

The or each sensor may include an analog to digital (A to D) converter for converting analog data to a digital domain. The A to D converter may be configured to convert an analog output from the or each sensor to the data prior to storing the data. The apparatus may include means for providing feedback to a subject being monitored.

The processor may be configured to execute an algorithm for evaluating a dynamic signature or change in dynamic signature of a body or body part(s) or joints. The algorithm may be adapted to evaluate the change in dynamic signature based on methods for comparing or evaluating a change in dynamic status.

In one form the processor may be adapted to provide a change in dynamic status Sₙ according to the following equation:

Sₙ=|Aₙ−A₀|

wherein:

“A₀” represents a control reference for the dynamic status of the body or body part which may include a baseline measurement (eg. the first measurement of dynamic status taken for the subject) at time t=0, or may be a normative value for a group of subjects (such as a team of athletes) or may represent indicative values of a physical quantity such as Peak, Root Mean Square (RMS) or Average of the or each physical quantity.

“Aₙ” represents a measurement taken at time t=n (wherein n≠0).

In one form Sₙ=100*|Aₙ−A₀|/|A₀|. Aₙ may represent the dynamic status of the body or body part at time t=n and A₀ may represent the control reference.

Relative change in samples of Aₙ may be defined as S_{Δn}. S_{Δn} may be visually represented via a graph with a trend line or may be compared with a pre-determined threshold. S_{Δn} may be used to classify a movement pattern as abnormal or normal.

The algorithm may be adapted to filter rotation data by applying a filter such as a band-pass filter. The algorithm may be adapted to transform data from a first frame of reference relative to a second frame of reference in which the body part performs a movement. For example the algorithm may be adapted to compensate for tibial angle to provide accelerations in a global frame. Steps of sensor data processing may include:
1) Filtering of Gyro data
2) Gyro integration in three dimensions
3) Transformation of tibial (bone of the limb) angle to a frontal plane. For example, it may be 45 degrees for a tibia of a human, or metatarsal for equus cabellus.
4) Integrated Gyro data may be used for transformation of 3D acceleration data from a Sensor to a Global frame

The algorithm may be adapted to integrate rotation and/or magnetic field data over a period of time to provide angular displacement. The algorithm may be adapted to integrate the data over a period of time to provide the angular displacement (⊖). The algorithm may be adapted to assemble the data over a period of time to provide a cluster of measurements or movements for an activity or for a range of activities. The algorithm may be adapted to evaluate a dynamic signature for the or each activity for a subject pre-injury. The algorithm may be adapted to store the dynamic signature for future reference, for example in the event that the subject is injured and requires rehabilitation. Following an injury the apparatus may take measurements to determine a dynamic signature of a body part. The apparatus may take further measurements to determine a dynamic signature of the body part during rehabilitation. The apparatus may compare measurements taken post injury and during rehabilitation, with the control signature to determine rehabilitation status of the body and/or fitness of a subject to return to active duty such as fitness of the sports-person to “return to play”.

The body part of the mammal may include legs and the apparatus may be adapted to monitor rotation components associated with the legs. Respective sensors may be applied to legs of the mammal. The or each sensor may include an analog to digital (A to D) converter for converting analog data to a digital domain. The A to D converter may be configured to convert an analog output from the or each sensor to the data prior to storing the data. Capturing angular deviation during dynamic lower extremity movements may require a sampling frequency that is at least sufficient and commensurate with frequency of the movement(s).

According to a further aspect of the present invention there is provided a method for monitoring, measuring and/or estimating dynamic status of a body part of a vertebral mammal, said method including:
using at least one kinematics sensor to measure relative to a first frame of reference data indicative of said dynamic status of said body part and for providing said data;
storing said data in a memory device; and
processing said data by a processor to evaluate a dynamic signature associated with said body part that correlates to said data.

### BRIEF DESCRIPTION OF DRAWINGS

FIG. 1 shows placement of sensors on the medial part of the tibia;

FIG. 2 shows one form of apparatus according to the present invention;

FIG. 3a shows a transversal plane cut of the tibia highlighting transformation of sensor data from sensor frame B to frame C;

FIG. 3b shows transformation of sensor data from frame C to global frame O;

FIG. 4 shows horizontal anterior-posterior accelerations and GRFs for one subject performing a deceleration test;

FIGS. 5a and 5b show scatter plots of slope of GRFs versus horizontal acceleration for two subjects performing a deceleration test;

FIGS. 6a and 6b show horizontal medio-lateral accelerations and GRFs for one subject performing a change of direction (COD) test for the right and left legs respectively; and

FIGS. 7a and 7b show scatter plots of medio-lateral accelerations and GRFs for two subjects performing a COD test.

### DETAILED DESCRIPTION

### Apparatus

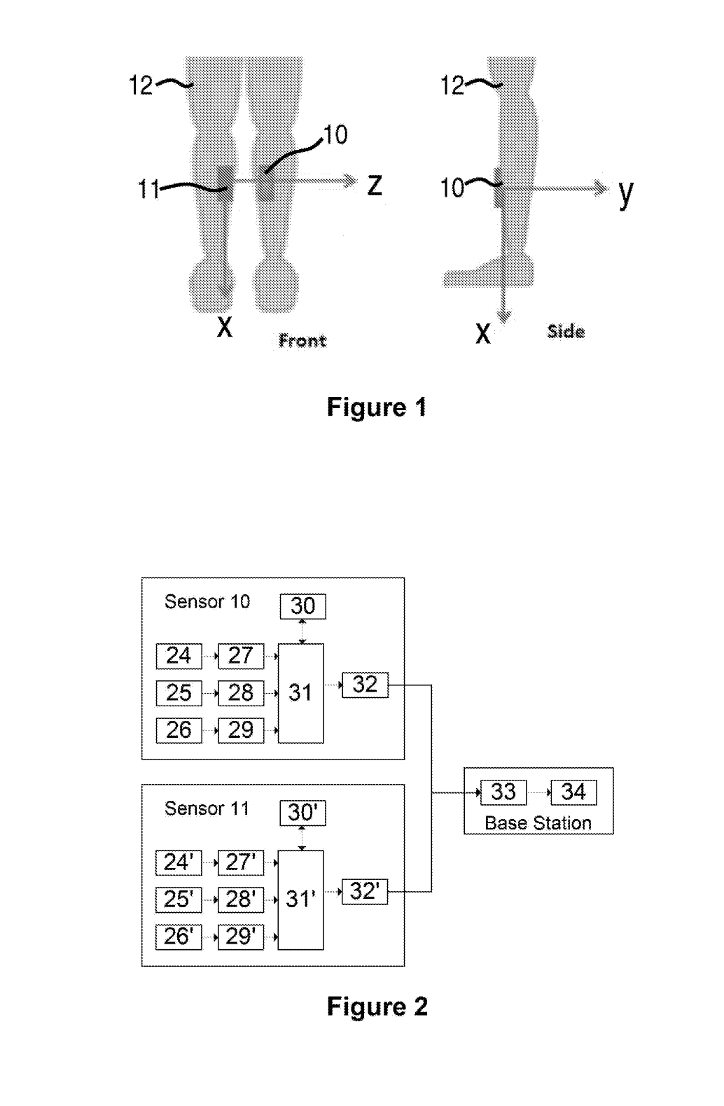

Apparatus according to the present invention may be placed on a body part such as a medial part of a tibia to enable monitoring of 3D dynamics as shown in FIG. 1. The apparatus may include acceleration sensors such as accelerometers and one or more inertial sensors such as gyroscopes and/or magnetometers as shown in FIG. 2. The apparatus may include a digital processing engine configured to execute one or more algorithms. The algorithm(s) may take account of variables such as movement of sensors during an activity relative to different frames of reference.

Referring to FIG. 1, one form of apparatus according to the present invention includes sensors 10, 11 placed along or in-line with tibial axes of the left and right legs of a human subject 12. Sensors 10, 11 are placed on the legs of subject 12 such that the frames of reference of sensors 10, 11 are defined by axes x,y,z with axes x,z being in the plane of FIG. 1 (front view) and axes x,y being in the plane of FIG. 1 (side view). For example measurement of Valgus or Varus may be defined as a rotation around the y axis.

Each sensor 10, 11 may include a rotation sensor such as a 1D, 2D or 3D gyroscope to measure angular velocity and optionally a 1D, 2D or 3D accelerometer to measure acceleration and/or a magnetic sensor such as a magnetometer to measure magnetic field. The positive axes on both legs may point up or down so that tibial acceleration may be measured in a vertical direction at least. Data from sensors 10, 11 may be used to ascertain a dynamic signature of the legs of subject 12 during activities and/or movements such as squatting, hopping and/or running.

Referring to FIG. 2 each sensor 10,11 includes sensor elements 24, 25, 26 and 24′, 25′, 26′ for measuring acceleration, angular rotation and magnetic field data respectively. Data obtained from sensors 24,25,26 and 24′,25′,26′ is converted from analog to digital format using Analog to Digital Converters (ADC) 27,28,29, and 27′, 28′, and 29′ respectively. The data may be stored in digital memories 30 and 30′ for analysis and reporting. Processing of signals is performed by Central Processing Units (CPUs) 31 and 31′. Sensor data measured via sensor elements 24, 25 and 26 and 24′, 25′ and 26′ may be sent via wireless transmitters 32, 32′ to remote receiver 33. Receiver 33 is associated with digital processing engine 34. Digital processing engine 34 includes a digital processor such as a microprocessor for processing data.

Digital memories 30, 30′ may include structure such as flash memory, memory card, memory stick or the like for storing digital data. The memory structure may be removable to facilitate downloading the data to a remote processing device such as a PC or other digital processing engine.

The digital memory 30, 30′ may receive data from sensor elements 24, 25, 26 and 24′, 25′, 26′. Each sensor element 24, 25, 26 and 24′, 25′, 26′ may include or be associated with a respective analog to digital (A to D) converter 27, 28, 29 and 27′, 28′, 29′. The or each A to D converter 27,28,29 and 27′,28′,29′ and memory 30, 30′ may be associated directly with sensor elements 24, 25, 26 and 24′, 25′, 26′ such as being located on the same PCB as sensor elements 24, 25, 26 and 24′, 25′, 26′ respectively. Alternatively sensor elements 24, 25, 26 and 24′, 25′, 26′ may output analog data to transmitters 32, 32′ and one or more A to D converters may be associated with remote receiver 33 and/or digital processing engine 34. The one or more A to D converters may convert the analog data to a digital domain prior to storing the data in a digital memory such as a digital memory described above. In some embodiments digital processing engine 34 may process data in real time to provide biofeedback to subject 12 being monitored.

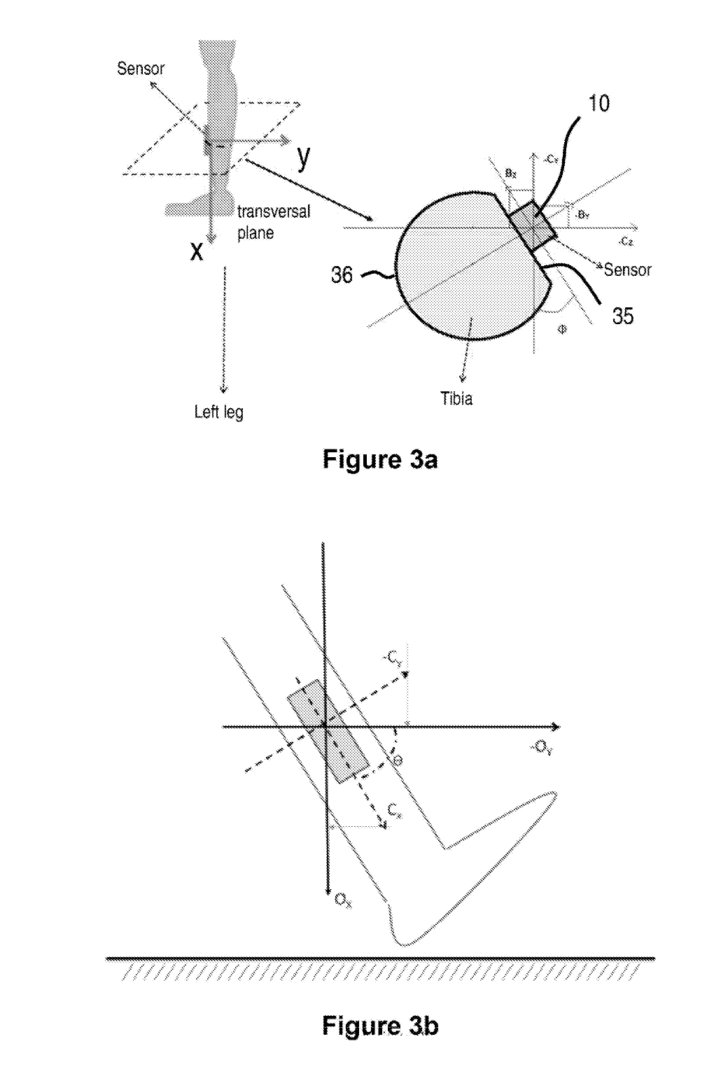

Digital processing engine 34 may include an algorithm for filtering and integrating gyroscope data, and transforming accelerations from a sensor element to a global frame perspective. Digital processing engine 34 may perform calculations with the algorithm to adjust for limb bone angle such as 45° for the tibia of a human being, following transformation of data from the frame of reference of each sensor 10 and 11 as shown in FIGS. 3a and 3b.

FIG. 3a shows a top-down cross-sectional view in the transversal plane of the left leg of subject 12 with sensor 10 placed on face 35 of tibia 36. The angle between face 35 on tibia 36 and the forward flexion plane is defined as φ. Angle φ may be approximately 45 degrees for an average subject but may vary a few degrees up or down from the average value. Face 35 may provide a relatively stable platform for attachment of sensor 10. The frame of reference (B) for sensor 10 is therefore rotated relative to the frame of reference (C) of the mechanical axis of tibia 36 by the magnitude of angle φ. Flexion and lateral flexion are defined as rotations around axes C_{Y} and C_{Z} while gyroscope and accelerometer sensitivity axes of sensor 10 are aligned with axes B_{Y} and B_{Z}.

Because measurements via sensor 10 are obtained in sensor reference frame B they must be converted to tibia reference frame C. The following equations may be used for this transformation:

Cy=By*cos(φ)+Bz*sin(φ) (1)

Cz=By*sin(φ)−Bz*cos(φ) (2)

wherein By, Bz denote y and z components in sensor reference frame B, Cy and Cz denote y and z components in tibia reference frame C, and φ denotes the angle between sensor 10 on tibia 21 and the forward flexion plane.

Equations (1) and (2) above may be used to vector transform gyroscope signals {^{B}ωₓ, ^{B}ω_{Y} and ^{B}ω_{Z}} and optionally accelerometer signals {^{B}aₓ, ^{B}a_{Y} and ^{B}a_{Z}} obtained via sensor 10 in sensor reference frame B, to gyroscope signals {^{C}ωₓ, ^{C}ω_{Y} and ^{C}ω_{Z}} and accelerometer signals {^{C}aₓ, ^{C}a_{Y} and ^{C}a_{Z}} respectively in mechanical or tibia reference frame C.

Following vector transformation, the gyroscope signals {^{C}_{ω}_{ₓ}, ^{C}ω_{Y} and ^{C}ω_{Z}} representing angular velocity may be integrated over a period of time t representing the duration of an activity such as squatting, hopping and/or running using the following equation to provide an integrated angular displacement (⊖):

⊖=∞₀^{t}ω, dt (3)

The integrated signals ⊖ may be corrected for gyroscope drift errors caused by noise and/or other artifacts. Drift correction may be performed using a known angular reference provided by the accelerometer signals. The flexion angle (⊖_{y}) may be corrected for drift at the start and at the end of a hop/squat using the flexion angle (β_{y}) obtained from the accelerometer signals using the following equation:

β_{y}a tan(^{C}a_{y}/^{C}aₓ) (4)

The lateral flexion angle (⊖_{Z}) may be corrected for drift using lateral flexion angle (β_{z}) obtained from the accelerometer using the following equation:

β_{z}a tan(^{C}a_{z}/^{C}aₓ) (5)

The twist angle (⊖_{X}) may be corrected with zero as there is no rotation around gravity measured by the accelerometer.

As a player flexes the knee, movement such as medio/lateral deviation is measured with respect to mechanical or tibia reference frame (C). However, this value is transformed with respect to the visual reference frame of the tester, also known as the frontal or viewer plane to provide more intuitive results.

It is possible for the leg to rotate around the x-axis when the player hops and lands. Hence, the visual impression of the lateral flexion will change if the rotation around the x-axis is not compensated. This effect is represented in equation 7, as it is used in the projection of the lateral flexion plane (⊖_{z}) with respect to the frontal plane.

FIG. 3a also shows a projection of lateral flexion angle (⊖_{Z}) onto the frontal or viewer plane together with a twist update. To project lateral flexion angle (⊖_{Z}) onto the frontal or viewer plane the leg may considered to be a rigid rod with fixed joint on the ankle. The length of the rod may be normalized as 1. Angular displacement on the ⊖_{X} plane (caused by ⊖_{Y} and ⊖_{Z} only) may be determined by:

⊖ₓ₀=a tan(sin(⊖_{Z})/tan(⊖_{Y})) (6)

Actual twist movement ⊖ₓ₁₀ may be added to angular displacement ⊖_{X} to determine resultant angular displacement ⊖_{Xresultant}:

⊖ₓᵣₑₛᵤₗₜₐₙₜ=Θₓ+Θₓ₀ (7)

One goal is to determine the terms A, B and C in order to calculate ⊖_{zAdjusted}. For this, the projection of ⊖_{Z} on ⊖_{X}, will result in A:

A=sin(⊖Z)/sin(⊖x0)*sin(⊖x) (8)

The projection of ⊖_{X} on ⊖_{Y} will determine B:

B=sin(⊖_{Z})/sin(⊖ₓ₀)*cos(⊖ₓ) (9)

C is calculated assuming the length of the rod is 1:

C=sqrt(1−B²) (10)

Finally, calculate a sin of A and C to obtain the drift adjusted ⊖_{Z} and projected onto the frontal plane as ⊖_{ZAdjusted}:

⊖_{ZAdjusted}=a sin(A/C) (11)

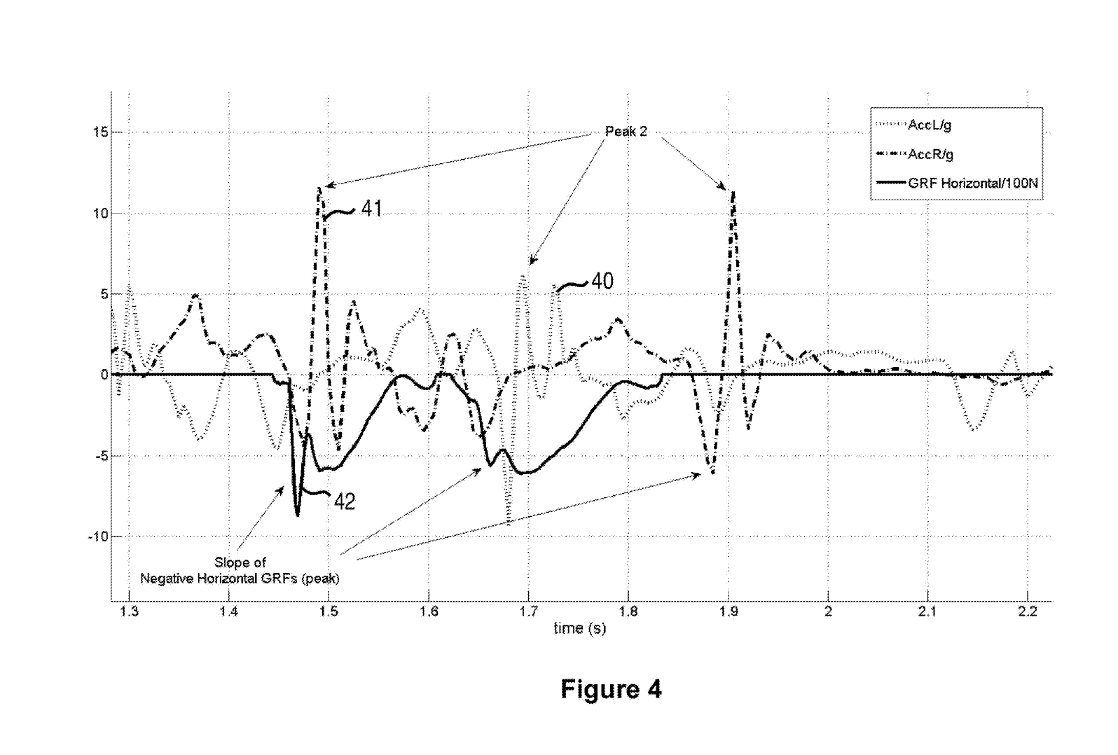

FIG. 4 shows test results for one subject performing a deceleration test. 3D accelerations are correlated with 3D GRFs. In FIG. 4, curve 40 represents horizontal anterior acceleration plotted over the duration of the test, while curve 41 represents horizontal posterior acceleration plotted over the same duration of the test. Curve 42 represents horizontal GRF plotted over the same duration of the test showing negative horizontal GRF. Curve 40 indicates that positive peak acceleration (acc_peak2) and the slope of horizontal GRF during the left leg stride shows less amplitude than the same variables measured during the right leg stride indicated by curve 41. Horizontal GRFs measured by a force plate or the like compared to anterior-posterior accelerations may provide information that accelerations are a valid measure of dynamic status of the limb. Anterior-posterior accelerations are compared with slope of horizontal GRFs as they occur in the same plane of reference and may be a more relevant kinematics variable to measure in a deceleration test, wherein the subject decelerates in the horizontal plane. Peaks of accelerations (for example, the initial peak acceleration of a foot colliding with the ground) may be representative of dynamic status of the lower limb during the active or stance phase of a stride.

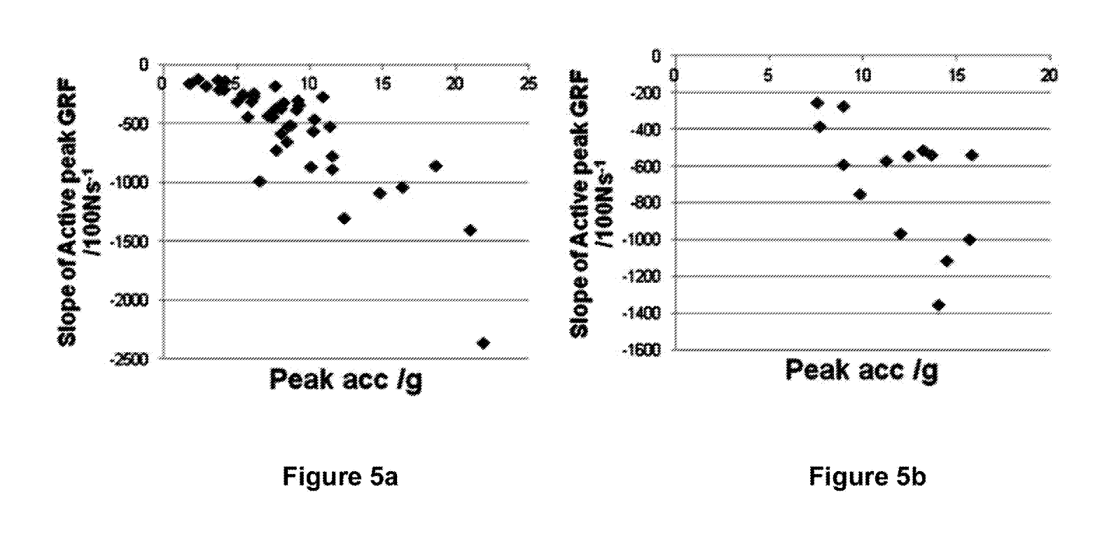

FIGS. 5a and 5b show test results for two subjects performing a deceleration test. 3D Accelerations are correlated with 3D GRFs. FIGS. 5a and 5b show scatter plots of slope of active peak GRF versus horizontal acceleration for subjects 1 and 2 respectively performing the deceleration test. FIGS. 5a and 5b show that there are strong correlations (>0.9) between the slope of horizontal GRF and horizontal accelerations when both subjects were forced to stop. Similarly this type of data may also be used to derive timing of run/test, cadence and/or load rates/peak accelerations during this, or other kinematic activities.

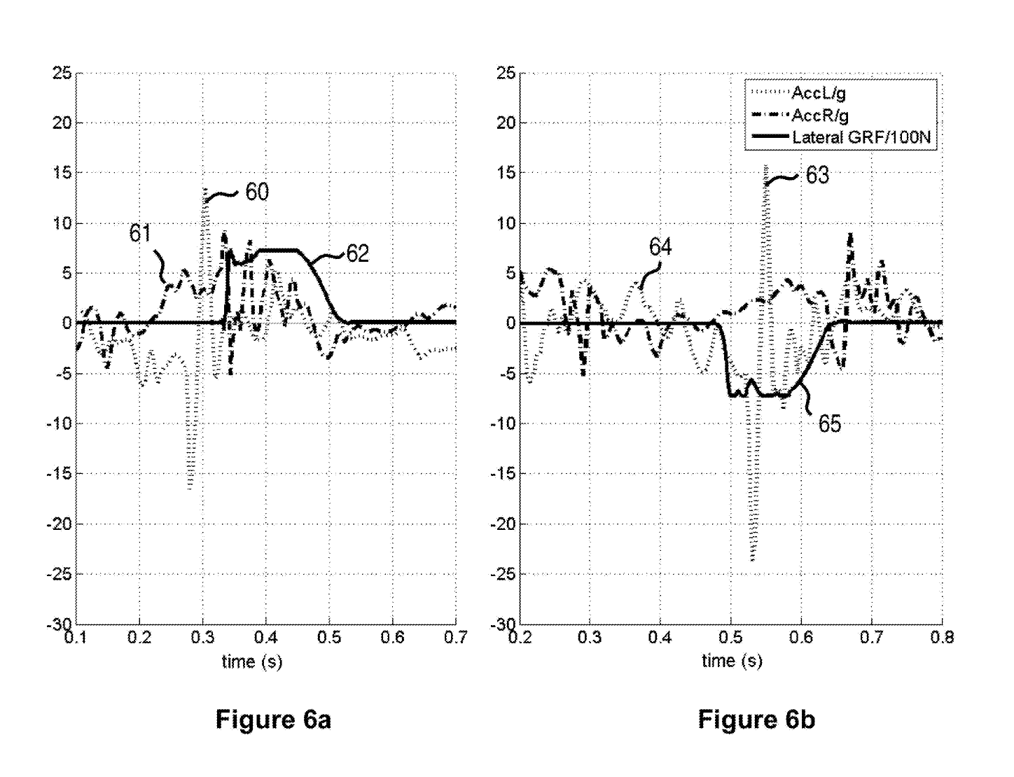

FIGS. 6a and 6b show test results for one subject performing a change of direction (COD) test. FIGS. 6a and 6b show plots of horizontal medio-lateral accelerations and GRFs for the change of direction (COD) test. 3D Accelerations are correlated with 3D GRFs. FIG. 6a shows the subject performing a one legged hop to the left and right for the right leg and FIG. 6b show the subject performing the one legged hop to the left and right for the left leg. FIG. 6 shows that the amplitude of lateral accelerations and lateral GRF during the subject's left leg hop (curves 63 and 65 respectively) showed higher amplitude than the ones measured on the right leg hop (curves 61 and 62 respectively) in the COD test. Lateral GRFs measured by a force plate or similar compared to lateral accelerations may provide information that accelerations are relevant kinematics variables to measure dynamic status of the limb during the COD test. Lateral accelerations are compared with lateral GRFs as they occur in the same plane of reference. Peaks of accelerations may be representative of dynamic status of the lower limb during the COD test.

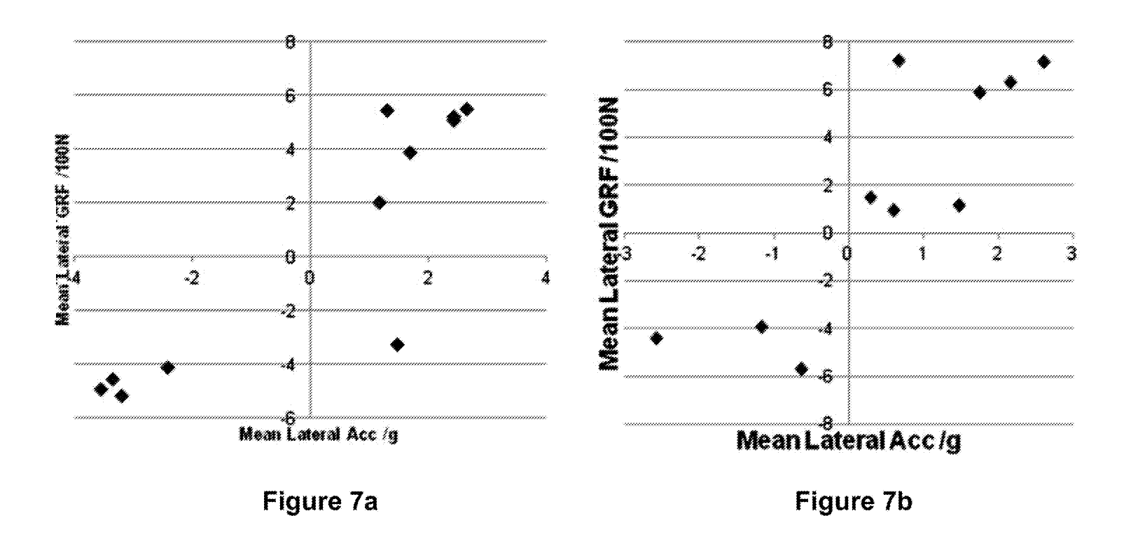

FIGS. 7a and 7b show test results for two subjects performing a change of direction (COD) test. FIGS. 7a and 7b show scatter plots of mean lateral GRF versus mean lateral accelerations for subjects 1 and 2 respectively. 3D Accelerations are correlated with 3D GRFs. FIG. 7a shows the scatter plots for subject 1 performing the COD test and FIG. 7b shows the scatter plots for subject 2 performing the COD test. FIGS. 7a and 7b show that there are strong correlations (>0.8) between the lateral GRFs and accelerations for both subjects in the COD test.

### Algorithms

Limb bone angle φ (such as 45 degree tibial angle for a human) is employed to change accelerations A and angular speeds Ω from sensor frame with tibia offset B to sensor frame C. It may be represented as a rotation matrix ^{C}_{B}M as:

A_{Cy}=A_{By}*cos(φ)+A_{Bz}*sin(φ)

A_{Cz}=A_{By}*sin(φ)−A_{Bz}*cos(φ)

Ω_{Cy}=Ω_{By}*cos(φ)+Ω_{Bz}*sin(φ)

Ω_{Cz}=Ω_{By}*sin(φ)+Ω_{Bz}*cos(φ)

filtered gyroscope data may be integrated over time→⊖_{C}=∞₀^{t} Ωc.dt, wherein Ω_{c} represents angular speed and ⊖_{c} represents angular displacement with respect to sensor frame C.

A rotation matrix ^{O}_{C}M may be defined to represent a matrix that translates a vector in sensor frame C to a global frame O. That is:

^{O}_{C}M ^{C}A=^{O}A

In this application, vector ^{C}A corresponds to accelerations measured with respect to sensor frame (C) being the frame aligned with the lower limb moving through 3D space in a forward direction but projected onto global frame (O) through the space.

Matrix ^{O}_{C}M embodies integrated gyroscope data ⊖_{C} as a Direct Cosine Matrix (DCM). This is shown in FIGS. 3a and 3b.

### EXAMPLES

### Deceleration Test

One or more sensors are fitted to a mammal on its lower limbs. Measurements may be taken as the mammal moves during a prescribed activity such as running over a pre-determined distance and/or stopping within a pre-determined distance causing deceleration. The measurement may be used to establish a control reference (signature of a movement pattern) constituted by speed, acceleration, stride rate (cadence) and/or load rate (newtons per time unit). Repeating the test and taking measurements as part of a routine test, check-up, onset of symptoms or following injury may be compared to a control reference or signature pattern considered to be normal (such as normative for a team) to assess dynamic status and/or change in the dynamic status. The data may also be used to rank the mammal and predict risk of injury (for example ranking players in a team).

### Joint Stability Test

One or more sensors are fitted to a mid-point of one or more lower limb/s of a mammal. As the mammal moves, lateral deviation of a joint during a sagittal plane flexion or extension (eg. knee joint of a human) may be measured. Lateral deviation, speed and other elements may also be measured during such dynamic activity. The measurements may indicate a weakness or instability in the joint. Measurements taken at one point in time may be used in the future as a reference to gauge the health or rehabilitation status of the joint being measured.

### Functional Test

One or more sensors are fitted to the mammal on the lower limbs and/or the joint connecting the lower limbs to the torso of the mammal. As the mammal moves during a prescribed activity of raising and lowering of the lower limbs, measurements of dynamic activity such as the limbs range of motion and how this affects the joint connecting to the torso are taken. How the torso is affected during such activities may indicate a weakness or deficiency in ligaments, joints and/or muscles used to perform the activity. Measurements taken at one point in time may be used in the future as a reference to gauge the health or rehabilitation status of the joints, ligaments and/or muscles being measured.

### Muscle Test

One or more sensors may be placed on the body or body part of a mammal and the sensor(s) monitors speed, velocity, range of movement and/or muscle activation of said part over one or multiple repetitions. The said part may be restricted (such as strapping down of a limb, splinted limb) or may be moving freely. The movement may be performed by the mammal or the mammal may be assisted to perform the movement. The data obtained may be used as a control reference and establish a signature of normal movement pattern. The protocol may be repeated at another time such as regular test or check-up, onset of symptoms or after injury and the data may be compared to the control reference and/or to a reference established to be normal (such as normative data from a team of players) to give indications on change in signature, abnormal movement pattern and/or risk of injury. This protocol may include comparisons between movements of a body part over time and/or movements of multiple body parts (such as one limb versus the other limb).

### Late Swing Phase Test

One or more sensors are fitted at a mid-point of one or more lower limb/s. As the mammal moves at a relatively fast pace, measurements are analysed relating to speed of the limb during a late phase swing, just prior to the limb striking the ground. Measurements include those relating to acceleration, velocity, angular rate of change and forces acting on the limb prior to and at the time of impact with the ground. Such measurements may then be compared to previous data being either normative or individual prior baseline data or reference data collected at an earlier time. The comparison may serve to indicate whether the measurements representing a current state of dynamic activity are similar to prior or reference data collected, and hence whether the current data is normal or abnormal.

Finally, it is to be understood that various alterations, modifications and/or additions may be introduced into the constructions and arrangements of parts previously described without departing from the spirit or ambit of the invention.

1. Apparatus for monitoring, measuring and/or estimating dynamic status of a body part of a vertebral mammal, said apparatus including:
at least one kinematics sensor for measuring relative to a first frame of reference data indicative of said dynamic status of said body part and for providing said data;
a memory device adapted for storing said data; and
a processor adapted for processing said data to evaluate a dynamic signature associated with said body part that correlates to said data.

2. Apparatus according to claim 1 wherein said kinematics sensor includes an acceleration sensor for measuring acceleration of said body part relative to said frame of reference and for providing data indicative of said acceleration.

3. Apparatus according to claim 2 wherein said acceleration sensor includes at least one inertial sensor.

4. Apparatus according to claim 1 wherein said kinematics sensor includes a rotation sensor for measuring rotation of said body part relative to said frame of reference and for providing data indicative of said rotation.

5. Apparatus according to claim 1 wherein said kinematics sensor includes a magnetic field sensor for measuring magnetic field around said body part and for providing data indicative of said magnetic field.

6. Apparatus according to claim 1 wherein said dynamic signature is measured prior to an injury to provide a control reference.

7. Apparatus according to claim 1 wherein said dynamic signature is measured following an injury to enable a material change in dynamic signature to be detected.

8. Apparatus according to claim 6 wherein said processor is adapted to execute an algorithm for evaluating a change in said dynamic signature of said body part relative to said control reference.

9. Apparatus according to claim 1 wherein said algorithm is adapted to transform said data from said first frame of reference to a second frame of reference in which said body part performs a movement.

10. Apparatus according to claim 1 wherein said algorithm is adapted to integrate said data over a period of time to provide an angular displacement (⊖).

11. Apparatus according claim 4 wherein said rotation sensor includes a gyroscope.

12. Apparatus according to claim 4 wherein said rotation sensor is adapted for measuring rotation around one or more orthogonal axes.

13. Apparatus according to claim 2 wherein said acceleration sensor is adapted for measuring acceleration along one or more orthogonal axes.

14. Apparatus according to claim 1 wherein said body part of said mammal includes legs and said apparatus is adapted to monitor rotation components associated with said legs.

15. Apparatus according to claim 1 wherein respective sensors are applied to the legs of said mammal.

16. Apparatus according to claim 1 wherein the or each sensor includes an analog to digital (A to D) converter for converting analog data to a digital domain.

17. Apparatus according to claim 16 wherein said A to D converter is configured to convert an analog output from the or each sensor to said data prior to storing said data.

18. Apparatus according to claim 1 including means for providing feedback of said deviation to a subject being monitored.

19. A method for monitoring, measuring and/or estimating dynamic status of a body part of a vertebral mammal, said method including:
using at least one kinematics sensor to measure relative to a first frame of reference data indicative of said dynamic status of said body part and for providing said data;
storing said data in a memory device; and
processing said data by a processor to evaluate a dynamic signature associated with said body part that correlates to said data.

20. A method according to claim 19 wherein said kinematics sensor includes an acceleration sensor for measuring acceleration of said body part relative to said frame of reference and for providing data indicative of said acceleration.

21. A method according to claim 20 wherein said acceleration sensor includes at least one inertial sensor.

22. A method according to claim 19 wherein said kinematics sensor includes a rotation sensor for measuring rotation of said body part relative to said frame of reference and for providing data indicative of said rotation.

23. A method according to claim 19 wherein said kinematics sensor includes a magnetic field sensor for measuring magnetic field around said body part and for providing data indicative of said magnetic field.

24. A method according to claim 19 wherein said dynamic signature is measured prior to an injury to provide a control reference.

25. A method according to claim 19 wherein said dynamic signature is measured following an injury to enable a material change in dynamic signature to be detected.

26. A method according to claim 24 wherein said processor executes an algorithm for evaluating a change in said dynamic signature of said body part relative to said control reference.

27. A method according to claim 19 wherein said algorithm is adapted to transform said data from said first frame of reference to a second frame of reference in which said body part performs a movement.

28. A method according to claim 19 wherein said algorithm is adapted to integrate said data over a period of time to provide an angular displacement (Θ).

29. A method according to claim 22 wherein said rotation sensor includes a gyroscope.

30. A method according to claim 22 wherein said rotation sensor is adapted for measuring rotation around one or more orthogonal axes.

31. A method according to claim 20 wherein said acceleration sensor is adapted for measuring acceleration along one or more orthogonal axes.

32. A method according to claim 19 wherein said body part of said mammal includes legs and said method includes monitoring rotation components associated with said legs.

33. A method according to claim 19 wherein respective sensors are applied to the legs of said mammal.

34. A method according to claim 19 wherein the or each sensor includes an analog to digital (A to D) converter for converting analog data to a digital domain.

35. A method according to claim 34 wherein said A to D converter is configured to convert an analog output from the or each sensor to said data prior to storing said data.

36. A method according to claim 19 including providing feedback of said deviation to a subject being monitored.
